(19) United States
(12) Patent Application Publication
(10) Pub. No.: US 2020/0029599 A1
(43) Pub. Date: Jan. 30, 2020

(54) CARBONATED DRINK AND METHOD OF MAKING SAME

(71) Applicant: Alex Getahoun, Norcross, GA (US)

(72) Inventor: Alex Getahoun, Norcross, GA (US)

(73) Assignee: Speed Laboratory, Inc., Norcross, GA (US)

(21) Appl. No.: 16/047,714

(22) Filed: Jul. 27, 2018

(51) Int. Cl. A23L 2/44; B67B 6/00; B67D 7/02; A23L 2/60; A23L 2/68; A23L 2/56; A23L 33/15; B67C 7/00; B67C 3/20; A61K 9/00; A61K 41/00; A61K 31/375

(52) U.S. Cl. CPC A23L 2/44; B67B 6/00; B67D 7/02; A23L 2/60; A23L 2/68; A23L 2/56; A23V 2002/00; B67C 7/00; B67C 3/208; A61K 9/0095; A61K 41/0004; A61K 31/375; A23L 33/15

(57) ABSTRACT

In a method of making a drink, a first predetermined quantity of a sweetener phase containing a base salt is dispensed into a bottle. A second predetermined quantity of an acidic aqueous phase is dispensed to the bottle. The bottle is sealed with a substantially airtight seal within a predetermined amount of time after the first predetermined quantity and the second predetermined quantity has been dispensed into the bottle. A fortified drink includes a first predetermined quantity of a sweetener phase, a second predetermined quantity of an acidic aqueous phase, a predetermined quantity of gesho extract and a pharmaceutically effective amount of an additive.

### BACKGROUND OF THE INVENTION

### 1. Field of the Invention

The present invention relates to carbonated drinks and, more specifically, to a method of making a carbonated drink containing an antimicrobial component.

### 2. Description of the Related Art

Carbonation of non-alcoholic beverages is commonly accomplished by forcing gaseous carbon dioxide (C0₂) into solution by applying sufficient pressure of CO₂ to the solution so as to result in dissolution of the CO₂ into the beverage during the packaging stage. In some alcohol-containing carbonated beverages (e.g., beer, sparkling wine, etc.), carbonation is achieved as part of the fermentation process. For fountain drinks, previously-carbonated water is combined with a flavoring syrup at the nozzle dispensing the drink.

Carbonization of certain liquids (e.g., antacid tablets such as Alka-Seltzer, etc.) is achieved by dissolving a tablet containing acid salts and base salts in water. In such tablets, the acid and base salts are of an appropriate composition to generate CO₂ when they are dissolved. Such liquids are generally not considered flavorful and are typically not consumed as beverages, but used as remedies for discomforts.

Recently, several companies have sold drink products that include water and flavor components that are fortified with vitamins. While many people prefer carbonated drinks, such vitamin-fortified drink products are not carbonated. This is because precise control of the chemical characteristics of the resulting solution, such as pH, necessary to maintain the efficacy of vitamins is difficult when pressurizing water with gaseous C0₂.

Therefore, there is a need for a carbonated drink including active ingredients with a predictable chemical environment.

### SUMMARY OF THE INVENTION

The disadvantages of the prior art are overcome by the present invention which, in one aspect, is a method of making a drink, in which a first predetermined quantity of a sweetener phase containing a base salt is dispensed into a bottle. A second predetermined quantity of an acidic aqueous phase is dispensed to the bottle. The bottle is sealed with a substantially airtight seal within a predetermined amount of time after the first predetermined quantity and the second predetermined quantity has been dispensed into the bottle. The acid or base dispensing component order may be reversed.

In another aspect, the invention is a method of making a fortified drink, in which a first predetermined quantity of a sweetener phase containing a base salt is dispensed into a bottle. A second predetermined quantity of an acidic aqueous phase is dispensed to the bottle. An effective amount of a gesho extract is dispensed into the bottle as a preservative. A pharmaceutically effective amount of an additive selected from a list of additives consisting of: a nutritional supplement, a vitamin, a medication, a homeopathic supplement and combinations thereof; is dispensed into the bottle. The bottle is sealed with a substantially airtight seal within a predetermined amount of time after each dispensing step.

In yet another aspect, the invention is a fortified drink that includes a first predetermined quantity of a sweetener phase, a second predetermined quantity of an acidic aqueous phase, a predetermined quantity of gesho extract and a pharmaceutically effective amount of an additive. The first predetermined quantity of a sweetener phase includes a base salt. The base salt includes a weight percent of the drink in a range of from 0.10% to 5.20%. The second predetermined quantity of an acidic aqueous phase includes an acid having a weight percent of the drink in a range of from 0.06% to 5.22%. The predetermined quantity of gesho extract is in an amount effective to act as a preservative. The pharmaceutically effective amount of an additive is selected from a list of additives consisting of: a nutritional supplement, a vitamin, a medication and a homeopathic supplement. The fortified drink is carbonated as a result of the sweetener phase reacting with the acidic aqueous phase after having been sealed in a bottle.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a schematic diagram demonstrating one method of producing a drink.

FIGS. 2A and 2B are graphs shown results of an antimicrobial effectiveness study demonstrating the effectiveness of gesho extract.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Also, as used herein, “gesho” means a plant of the R. prinoides species, which is also sometimes referred to as “shiny-leaf buckthorn” and “medium coarseness” means having a mean particle size in a range of between 0.5 mm² to 9 mm².

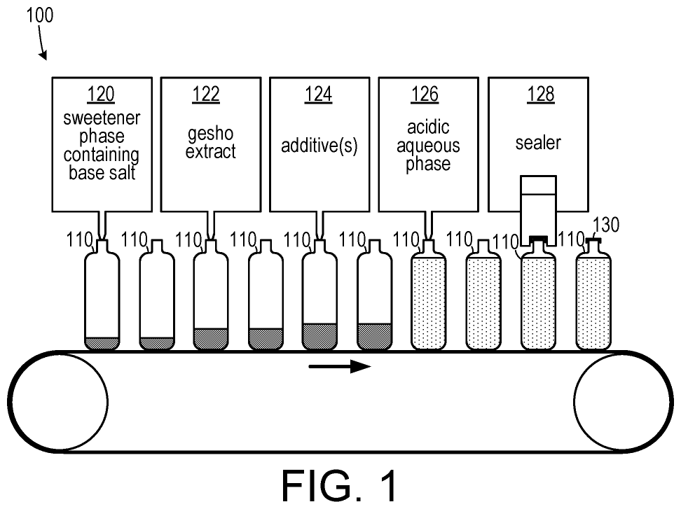

As shown in FIG. 1, one method 100 of making a drink includes dispensing a first predetermined quantity of a sweetener phase containing a base salt 120 into a bottle 110. The sweetener phase can include a solution of water and a sweetener such as, for example: honey, maple syrup, corn syrup, high fructose corn syrup, agave nectar, dissolved sugar, or any one of many sweeteners known to the food sciences art. The base salt can include, for example, a substance selected from a list consisting of: potassium hydrogen carbonate; sodium carbonate; potassium carbonate; magnesium carbonate; sodium hydrogen carbonate; and calcium carbonate. Depending on the amount of carbonation desired, the amount of the base salt added is in a weight percent of the drink in a range of from 0.10% to 5.20% of the final weight of the drink. Typically, the sweetener phase /base salt 120 will be dispensed as a viscous fluid having a viscosity similar to that of honey or molasses. This phase can be diluted with water to reduce viscosity. An effective amount of a gesho extract 122 is added into the bottle and acts as a preservative. The gesho extract 122 also adds flavor to the drink. In one embodiment, additives 124, such as a pharmaceutically effective amount of active ingredients (for example: a nutritional supplement, a vitamin, a medication, a homeopathic supplement) can be added. Other additives that can be added include flavor additives (for example, grape, cherry, lemon, lime additives, etc.) and coloring agents (e.g., food coloring agents).

Immediately prior to sealing the bottle 110, a quantity of an acidic aqueous phase 126 is added to the bottle. The acidic aqueous phase 126 can include a solution in water of a substance such as: citric acid; malic acid; maleic acid; fumaric acid; ascorbic acid; and tartaric acid. Depending upon the amount of base salt used, the acidic aqueous phase includes water and an acid. In one embodiment, the amount of an acid that has a weight percent of the drink in a range of from 0.06% to 5.22%.

The bottle 110 is sealed with a sealing device 128 within a predetermined amount of time after acidic aqueous phase 126 has been added. The acid in the acidic aqueous phase 126 reacts with the base salts to release carbon dioxide into the drink. Therefore, the bottle 110 should be sealed with an airtight seal 130 before the reaction is complete so that most of the carbon dioxide produced by the reaction will remain in the drink after the bottle 110 is sealed. The airtight seal 130 can include any one of the many drink sealing devices known to the art, including press-on bottle caps, screw-on bottle caps and the like.

Because the amount of the carbon dioxide that remains in the drink is a function of the amount of reactants used and the timing of the sealing of the bottle 110, this amount can be controlled precisely by controlling the process.

Generally, a basic sweetener phase (sweetener containing a base salt), is first dispensed into a bottle, with an acidified and sometimes flavored aqueous phase following, then a closure is immediately applied. Without agitation, the basic sweetener phase will react with the acidic aqueous phase to produce CO₂ in solution; if agitated, C0₂ production is faster. For fountain drinks, the standard equipment currently used is still suitable for dispensing both the basic sweetener phase (replacing the flavored syrup), and the acidic and flavored aqueous phase (replacing the carbonated water).

The gesho extract can be made by grinding at least one of sticks and/or leaves of the gesho plant (R. prinoides) to a medium coarseness to form gesho particles. Grinding the particles too finely can result in premature clogging of the filtering media used in the process and grinding them too coarsely can result too much time being taking in leaching the extract from the particles. In on embodiment, the particles will have an average diameter in a range of about 1 mm to 3 mm. Ethanol or an ethanol/water mixture is added to the gesho particles in an amount sufficient to dissolve a predetermined amount of soluble gesho material from the gesho particles. (In certain embodiments, water may be used as a solvent) The ethanol and gesho particles are agitated sufficiently to maintain the gesho particles in suspension for up to about eight hours, thereby generating a gesho extract/ethanol solution. The gesho extract/ethanol solution is then filtered, thereby separating the gesho extract/ethanol solution from the now-depleted gesho particles. The gesho extract/ethanol filtrate solution is concentrated so as to generate a viscous gesho liquid by subjecting the gesho extract/ethanol to a rotary vacuum concentrator (for example, a Savant™ SpeedVac™ High Capacity Concentrators available from T Thermo Fisher Scientific Inc.) for a predetermined amount of time at a predetermined temperature. Vacuum is applied and flask rotation is started. The rotary vacuum concentrator includes a flask into which the gesho extract/ethanol solution is dispensed and a condenser. In one embodiment, the flask is heated to about 35° C. and the condenser is maintained at a temperature of about −78° C. Then the flask is cooled to about −8° C. during the concentrating step. The viscous gesho liquid is vacuum dried until the anti-microbial extract has a predetermined dryness. In alternative embodiments, the gesho liquid can be either freeze dried or spray dried. In certain embodiments, if the extract is freeze dried, it can be tried at a temperature range of 0° C.-120° C.±5° C. at a vacuum of between atmospheric pressure down to about 0.4 Torr for 8-48 hours (depending on batch size). If spray drying is used, in certain embodiments it may be done at an air temperature in a range of Air temp at 160° C. to 200° C.±20° C. with a Feed Rate of 5-50 mL/min.

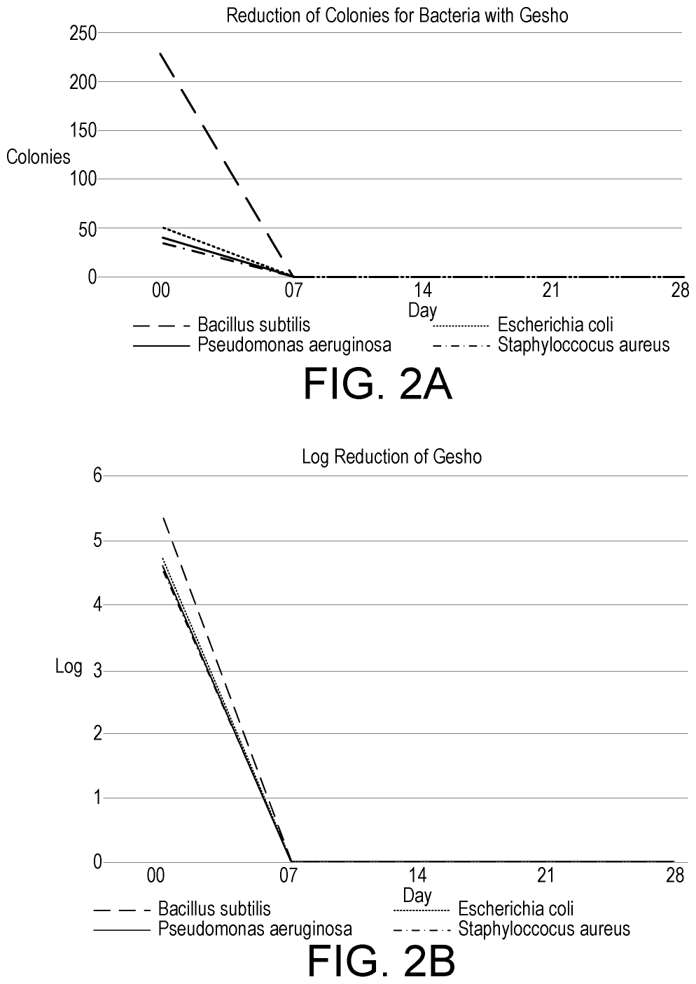

Applicant has demonstrated that the gesho extract exhibits antimicrobial activity and, therefore, it can be used both as a preservative in the sweetener phase. Applicant conducted a study to demonstrate antimicrobial efficiency of gesho extract based on the United States Pharmacopeia (USP) chapter <51>, an antimicrobial effectiveness test (AET), which was performed at Speed Laboratory Incorporated, Norcross, Ga. in June of 2018. In the study, the gesho extract was tested against cultures of the following microbes: Bacillus subtilis, Pseudomonas aeruginosa, Escherichia coli, and Staphyloccocus aureus. As shown in FIGS. 2A and 2B, this study has demonstrated that the gesho extract, as produced by the above-described method, demonstrates substantial antimicrobial effectiveness within seven days against each of these species.

In one experimental embodiment, a general range (weight to volume) for the acid component in the finished product would be from approximately 0.06% to 5.22%, with a preferred target amount of from approximately 0.63% for maleic acid, to a high of 1.74% for tartaric acid. (The theoretical high acid component would be ascorbic acid, but that amount would exceed the amount that is normally tolerable by the digestive system, and therefore, ascorbic acid is generally maintained at a recommended maximum 0.500 g.) The sweeteners are added at a rate of between 0.5% to 8.0%. With a preferred target amount of between 3.0% to 6.0% depending desired sweetness and overall flavor profile. Flavoring components are added over the range of 0.05% up to about 1.0%. Gesho preservative was added at about 0.01% up to 5.0%. When acid-base reactions were balanced the preferred target amount of carbon dioxide is calculated at 0.124 moles per liter based on molecular weight. The amount of carbonation can be increased or decreased to meet personal preferences.

The specific amounts of individual or combined acid and base compounds can be varied depending on the specific acid-base reactions that are involved and are calculated based on those specific reactions (acid/base equivalents). The base component determines how much carbonation (C0₂) is available and the acid component is then determined by balancing the respective chemical equation so that one equivalent of acid is present for each equivalent of base; it is not critical that the equation is exactly numerically balanced, but that a close approximation (±1-2%) will produce reasonably consistent and acceptable results for both carbonation and taste. Necessarily, the base component must be a compound capable of providing at least one C0₂ moiety.

The number of equivalents for each base compound can be as follows: Sodium hydrogen carbonate -one (1), potassium hydrogen carbonate -one (1), sodium carbonate -two (2), potassium carbonate -two (2), magnesium carbonate -two (2), and calcium carbonate -two (2).

The number of equivalents for each acid compound can be as follows: ascorbic acid -two (2), citric acid three (3), malic acid -two (2), maleic acid -two (2), fumaric acid -two (2), and tartaric acid -two (2). All percentages are stated for final product total. A general range (weight to volume) for the base component in the finished product would be from approximately 0.10% to 5.20%, with a preferred target amount of from approximately 1.04% for Sodium Hydrogen Carbonate, to a high of 1.71% for Potassium Carbonate.

In one experimental embodiment of a drink, the following formulation was used:
Honey 20 g (5.63%)
Sodium Hydrogen Carbonate 3.70 g (1.04%)
Ascorbic Acid 0.50 g (0.14%)
Citric Acid 2.46 g (0.69%)
Gesho Extract 1.78 g (0.50%)
Grape Flavor 0.53 g (0.15%)
Water 329 mL Approximately 355 mL final volume

In this experimental embodiment, the specified amount of base component was incorporated into the sweetening component. The corresponding amount of acid components were dissolved into water. The preservative was added to the water/acid phase and stirred until dissolved. The flavor component was added to the water/acid/preservative solution. B Vitamins were added to the water/acid/preservative/flavor.

In one experimental embodiment dried gesho leaf was ground to medium coarseness. About 50 grams of ground gesho leaf was placed into a 500 ml Erlenmeyer flask and about 250 mL of 190 Proof (95%) grain alcohol (ethanol) was added to the flask. The vessel was orbitally shaken at approximately 150 rpm for about 72-96 hours. The resulting extracted material was vacuum filtered through a 20 um fast filter (using a paper filter medium). The filtrate was placed into a flask of suitable size and attached to a rotary vacuum concentrator. A vacuum was applied and flask rotation is started. The flask was heated to approximately 35° C., and the condenser was held at approximately −78° C. This reduced the extract to minimal liquid (e.g. about 10-20 mL), then the flask was cooled to approximately −8° C. The process continued until the condenser no longer produced a continuous drip and the product was a thick viscous consistency. It was found that the rotary vacuum drying process typically completes in about 8 hrs.

For a bottled product, the base/sweetener component was dispensed into the bottle. The water/acid/preservative/flavor/vitamin component was then added to the bottle and the bottle was immediately capped. In a soft drink dispensing embodiment, the base/sweetener component (syrup) replaces the flavoring syrup. The water/acid/preservative/flavor component replaces the carbonated water.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. 112(f) unless the words “means for” or “step for” are explicitly used in the particular claim. The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A method of making a drink, comprising the steps of:
(a) dispensing a first predetermined quantity of a sweetener phase containing a base salt into a bottle;
(b) dispensing a second predetermined quantity of an acidic aqueous phase to the bottle; and
(c) within a predetermined amount of time after the first predetermined quantity and the second predetermined quantity has been dispensed into the bottle, sealing the bottle with a substantially airtight seal.

2. The method of claim 1, further comprising the step of dispensing an effective amount of a gesho extract into the bottle prior to the sealing step as a preservative.

3. The method of claim 1, wherein the base salt comprises a substance selected from a list consisting of: potassium hydrogen carbonate; sodium carbonate; potassium carbonate; magnesium carbonate; sodium hydrogen carbonate; and calcium carbonate.

4. The method of claim 1, wherein the acidic aqueous phase comprises a solution in water of a substance selected from a list consisting of: citric acid; malic acid; maleic acid; fumaric acid; ascorbic acid; and tartaric acid.

5. The method of claim 1, further comprising the step of dispensing into the bottle prior to the sealing step a pharmaceutically effective amount of an additive selected from a list of additives consisting of: a nutritional supplement, a vitamin, a medication, a homeopathic supplement and combinations thereof.

6. The method of claim 1, wherein the base salt comprises a weight percent of the drink in a range of from 0.10% to 5.20%.

7. The method of claim 1, wherein the acidic aqueous phase comprises an acid having a weight percent of the drink in a range of from 0.06% to 5.22%.

8. The method of claim 1, further comprising the step of dispensing at least one flavor additive into the bottle prior to the sealing step.

9. The method of claim 1, further comprising the step of dispensing at least one coloring agent into the bottle prior to the sealing step.

10. A method of making a fortified drink, comprising the steps of:
(a) dispensing a first predetermined quantity of a sweetener phase containing a base salt into a bottle;
(b) dispensing a second predetermined quantity of an acidic aqueous phase to the bottle
(c) dispensing an effective amount of a gesho extract into the bottle as a preservative;
(d) dispensing into the bottle a pharmaceutically effective amount of an additive selected from a list of additives consisting of: a nutritional supplement, a vitamin, a medication, and a homeopathic supplement; and
(e) within a predetermined amount of time after each dispensing step, sealing the bottle with a substantially airtight seal.

11. The method of claim 10, wherein the base salt comprises a substance selected from a list consisting of: potassium hydrogen carbonate; sodium carbonate; potassium carbonate; magnesium carbonate; sodium hydrogen carbonate; and calcium carbonate.

12. The method of claim 10, wherein the acidic aqueous phase comprises a solution in water of a substance selected from a list consisting of: citric acid; malic acid; maleic acid; fumaric acid; ascorbic acid; and tartaric acid.

13. The method of claim 10, wherein the base salt comprises a weight percent of the drink in a range of from 0.10% to 5.20%.

14. The method of claim 10, wherein the acidic aqueous phase comprises an acid having a weight percent of the drink in a range of from 0.06% to 5.22%.

15. The method of claim 10, further comprising the step of dispensing at least one flavor additive into the bottle prior to the sealing step.

16. The method of claim 10, further comprising the step of dispensing at least one coloring agent into the bottle prior to the sealing step.

17. The method of claim 10, wherein the gesho extract is made by the steps of:
(a) grinding gesho matter to a medium coarseness to form gesho particles;
(b) adding an amount of ethanol sufficient to dissolve a predetermined amount of soluble gesho material from the gesho particles;
(c) agitating the ethanol and gesho particles sufficiently to maintain the gesho particles in suspension for a predetermined amount of time, thereby generating a gesho extract/ethanol solution;
(d) after the agitating step, vacuum filtering the gesho extract/ethanol solution, thereby separating the gesho extract/ethanol solution from the gesho particles;
(e) concentrating the gesho extract/ethanol solution so as to generate a viscous gesho extract liquid; and
(f) vacuum drying the gesho extract liquid to a predetermined dryness.

18. A fortified drink, comprising:
(a) a first predetermined quantity of a sweetener phase including a base salt, wherein the base salt includes a weight percent of the drink in a range of from 0.10% to 5.20%;
(b) a second predetermined quantity of an acidic aqueous phase wherein the acidic aqueous phase includes an acid having a weight percent of the drink in a range of from 0.06% to 5.22%;
(c) a predetermined quantity of gesho extract in an amount effective to act as a preservative; and
(d) a pharmaceutically effective amount of an additive selected from a list of additives consisting of: a nutritional supplement, a vitamin, a medication and a homeopathic supplement,
the fortified drink being carbonated as a result of the sweetener phase reacting with the acidic aqueous phase after having been sealed in a bottle.

19. The fortified drink of claim 18, wherein the base salt comprises a substance selected from a list consisting of: potassium hydrogen carbonate; sodium carbonate; potassium carbonate; magnesium carbonate; sodium hydrogen carbonate; and calcium carbonate.

20. The fortified drink of claim 18, wherein the acidic aqueous phase comprises a solution in water of a substance selected from a list consisting of: citric acid; malic acid; maleic acid; fumaric acid; ascorbic acid; and tartaric acid.
